(19) United States
(12) Patent Application Publication
(10) Pub. No.: US 2012/0004645 A1
(43) Pub. Date: Jan. 5, 2012

(54) CAPSULAR URINARY CATHETER

(76) Inventor: Mohamad Dastani, Esfahan (IR)

(21) Appl. No.: 12/830,288

(22) Filed: Jul. 3, 2010

(51) Int. Cl. A61F 5/453; A61F 5/455; A61M 27/00

(52) U.S. Cl. 604/544; 604/350; 604/347

(57) ABSTRACT

The various embodiments herein provide a capsular urinary catheter to manage urinary incontinence or retention in patients to prevent the Catheter Associated Urinary Tract Infections in patients. The urinary catheter comprising a first end adapted to be inserted into the patient's body, a second end adapted to be releasably attached to a fluid collection reservoir, a body defining an exterior surface and an interior surface, a vaginal condom segment attached to the exterior surface, a capsular segment defining a capsule in the interior surface and three one way valves arranged inside the capsular segment. Each one way valve includes at least two plates which gets separated due to a force exerted by a fluid movement, thereby causing the passage of a fluid from the first end towards the second end and prevents a fluid reflux to the body of the patients.

The present invention is sponsored by Iranian National Science Foundation for international filing.

### BACKGROUND

1. Technical Field

The embodiments herein generally relates to urinary catheters and particularly to a urinary catheter for draining bladder to manage urinary incontinence in patients. The embodiments herein more particularly relates to a urinary catheter which prevents urinary reflux and microorganisms entering into the human body.

2. Description of the Related Art

Catheters and other devices are used in draining of accumulated fluids from the human body or injecting fluids into the body and have been widely used in the medical field for a long period of time. In particular, with reference to catheters, there are numerous constructions and designs, each of which is particularly adapted to be inserted into a particularly designated body cavity. For example, a urethral catheter is designed to drain accumulated urine secretions from the bladder.

Generally, urinary catheters are intended to permit continuous collection of urine of patients suffering from urinary incontinence or from disabled individuals. The field of urinary catheters has long been dominated by the Foley-type catheter, which is well known in the art. The Foley-type catheter comprises an inflatable balloon for disposition within a patient's bladder and a discharge or drainage tube which extends from the bladder, through the urethra, to the exterior when the catheter is in place within the patient. The Foley-type catheter provides passive urinary drainage, and the ability to clamp the catheter closed at a location exterior to the body of the patient.

The Foley-type catheters however have drawbacks such as relatively high intra-urethral leakage rates, the inability to selectively control discharge and the diminution in the patient's mobility or physical activities due to the constant need for a drainage collection device interconnected to the open catheter.

The use of an inflatable balloon or other blocking device to minimize intra-urethral leakage around the exterior of the catheter and valves disposed within the catheter body to permit selective voiding are considered fundamental advances in the art. However, these catheters are generally unsuitable for use by female patients and are subjected to leakage resulting from normal body movement when used in male patients.

A wide variety of discharge valve designs have been contemplated for use with urinary catheters. These valves are conventionally actuated mechanically, but may also be actuated magnetically or by other suitable means. The more prevalent types of mechanical valves include ball-and-seat, duckbill, inflatable check, plug, and dome-type valves. These valves may all be operated manually by the patient, however, some types of valves are identified as “palpitatable” based upon a portion of the valve being squeezed or pressed in order to open the valve. These palpitatable valves are more difficult to use, uncomfortable, and unreliable.

Hence there is a need to provide a urinary catheter which prevents the flow of drained material to re-enter the body of the patient. There also exists a need to provide a urinary catheter to manage urinary incontinence or retention for preventing Catheter Associated Urinary Tract Infections (UTI) in patients. Furthermore, there exists a need to provide a urinary catheter which can be used in both female patients and male patients.

The abovementioned shortcomings, disadvantages and problems are addressed herein and which will be understood by reading and studying the following specification.

### OBJECTIVE OF THE EMBODIMENTS

The primary object of the embodiments herein is to provide a urinary catheter that does not allow the flow of drained material to re-enter the body of the patient.

Another object of the embodiments herein is to provide a urinary catheter to prevent Catheter Associated Urinary Tract Infections (UTI).

Yet another object of the embodiments herein is to provide a urinary catheter device which can be employed in both female patients and male patients.

Yet another object of the embodiments herein is to provide a urinary catheter with one way cone shaped valves to prevent urinary reflux and microorganisms entering into the patient's urinary bladder.

Yet another object of the embodiments herein is to provide a urinary catheter with a vaginal condom segment to connect the urinary catheter to the glands of penis in men and vaginal opening in women.

Yet another object of the embodiments herein is to provide a urinary catheter with a stopper mechanism to prevent separation of the vaginal condom segment from the urinary catheter.

Yet another object of the embodiments herein is to provide a urinary catheter with automatic valves to eliminate patient's intervention for fluid regulation.

Yet another object of the embodiments herein is to provide a urinary catheter adapted to vary its length based on the sexuality of the patients.

These and other objects and advantages of the embodiments herein will become readily apparent from the following detailed description taken in conjunction with the accompanying drawings.

### SUMMARY

The various embodiments herein provide a urinary catheter to manage urinary incontinence or retention in patient for preventing urinary reflux thereby avoiding microorganism re-entering into patient's urinary bladders. The urinary catheter comprising a first end adapted to be inserted into the patient's body, a second end adapted to be releasably attached to a fluid collection reservoir, a body defining both an exterior surface and an interior surface, a vaginal condom segment attached to the exterior surface, a capsular segment defining a capsule in the interior surface, three one way valves arranged inside the capsular segment. Each of the three one way valves includes two plates which gets separated due to a force exerted by a fluid movement, thereby causing the passage of fluid from the first end towards the second end and prevents fluid reflux from the second end towards the first end of the catheter.

According to an embodiment, the three one way valves are arranged within lumen of catheter, a vaginal-condom segment on the exterior surface of the catheter, a stopper is fixed on to the vaginal-condom segment of the catheter to prevent the disconnection of vaginal-condom from patients body. The three one way valves that are located within the capsular segment of the urinary catheter are of cone shaped. Each of the cone shaped valves is made of two pieces of separable plastic that stick together pointing towards the second end of the urinary catheter.

According to an embodiment of the present disclosure, the interior surface defines a central lumen through which urine flows from a urinary bladder to the fluid collection reservoir.

According to an embodiment of the present disclosure, the three one way valves comprise an infundibular valve floor extending towards the first end of the catheter and a valve tip extending towards the second end of the catheter. The valve tip includes at least two seperably attached plates tapering towards the second end of the urinary catheter. As the valves are made of two pieces of separable and flexible plastic, the force and weight of the urine entering into the capsular segment results in opening of valves thereby allowing the urine to flow through the valves.

According to an embodiment of the present disclosure, the two plates of the valve tip separate from each other due to a force exerted by the urine flow from the urinary bladder thereby opening the three one way valves to direct the urine flow to the collection reservoir. The two plates stick together due to the elasticity of the material resulting in the closure of a fluid flow route thereby preventing the urine reflux. Here, the three one way valves are arranged equidistantly within the capsular segment.

According to an embodiment of the present disclosure, the vaginal condom segment is adapted to connect the urinary catheter with glands of penis in men and around the vagina in women. The vaginal condom segment comprises of a crescent segment arranged adjacent to a beginning part of the vaginal condom segment and a flexible arm. The flexible arm includes a plurality of folds which provides for varying the length of the flexible arm in proportionate with sexuality of the patients.

According to an embodiment of the present disclosure, the design of the vaginal condom segment is such that it is used in connecting the urinary catheter to male and female patients. Further a tubular kind of material is used as a stopper which prevents the vaginal condom segment getting separated from the urinary catheter.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a top side perspective view of a capsular urinary catheter according to an embodiment of the present disclosure.

FIG. 2 illustrates a sectional view of the capsular segment and a three one way valves arranged in close contact position in a capsular urinary catheter according to an embodiment of the present disclosure.

FIG. 3 illustrates a sectional view and schematic view of one way valves inside the capsular segment in a capsular urinary catheter according to an embodiment of the present disclosure.

FIG. 4 illustrates a sectional view of the tip of the valve and the valve present in capsular segment of the urinary catheter according to an embodiment of the present disclosure.

FIG. 5 illustrates a perspective view of the valve cut into two equal halves in a capsular urinary catheter according to an embodiment of the present disclosure.

FIG. 6 illustrates a top view and a sectional view of the valve in the capsular segment of the urinary catheter according to an embodiment of the present disclosure.

FIG. 7 illustrates a perspective view of the vaginal condom segment in a capsular urinary catheter according to an embodiment of the present disclosure.

FIG. 8 illustrates a side cross sectional view of the vaginal condom segment in a capsular urinary catheter according to an embodiment of the present disclosure.

FIG. 9 illustrates a side cross sectional view of the vaginal condom segment with folds attached to the urinary catheter according to an embodiment of the present disclosure.

FIG. 10 is a schematic view of the urinary catheter illustrating a stopper which prevents detachment of vaginal condom segment from the urinary catheter according to an embodiment of the present disclosure.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a system and method for preventing catheter associated Urinary Tract Infections in patients using a urinary catheter device. A urinary catheterization, or “catheter” for short, is a plastic tube known as a urinary catheter that is gently slid into a patient's bladder via urethra. Catheterization allows to drain out the urine freely from the patients' bladder for collection, or to inject liquids used for treatment or diagnosis of bladder conditions. According to an embodiment herein, the urinary catheter comprises three one way valves within a capsular segment of the lumen of the urinary catheter, a vaginal-condom segment on the exterior surface of the catheter and a stopper fixed on to the vaginal-condom segment of the catheter to prevent the detachment of vaginal-condom segment from patients body.

The first end of the urinary catheter is inserted into the penis in male human body and into vagina in the female human body. The first end of the urinary catheter is a thin long plastic tubular pipe structure which extends from the urinary catheter to the patients bladder. The first end of the urinary catheter is connected using the vaginal condom segment present in the urinary catheter device. The vaginal condom segment has a unique design of folds present on a flexible arm which helps in connecting the catheter into the human body without any intervention. The length of the flexible arm can be varied manually depending upon the sexuality of patients as the flexible arm is made up of one or more folds. The vaginal condom segment includes a crescent segment located at the initial stage of the vaginal-condom segment facing the second end of the urinary catheter. The vaginal-condom segment is adapted to connect the urinary catheter to the glands of penis in men and around vagina in women. This process is accomplished with a sticky part on the edge of the convexity of the crescent segment of the vaginal-condom segment.

The vaginal condom segment is arranged such that the convexity of the crescent segment faces towards the second end of the crescent segment to attach the vaginal condom segment to the glands of penis in men. When the crescent segment is placed on the vaginal opening of the women, the concavity of the crescent segment face towards the second end and the edge of the convexity of the crescent segment is placed on the vaginal opening of the women.

Further the second end of the urinary catheter is connected to a urinary collection bag which is placed away from the urinary catheter and connected using a plastic tubular pipe.

Once the first end of the urinary catheter is connected to the urinary bladder, the urine starts flowing through the urinary catheter through a hollow passage way extending to the capsular segment of the urinary catheter. The capsular segment of the urinary catheter comprises of three one way cone shaped valves which are of single flexible or elastic material such as latex rubber or silicon. The three one way valves located in the capsular segment of the urinary catheter include a valve floor. The valve floor is of infundibular form and narrowing towards the second end of the urinary catheter. The valve tip is made of two separable plates made of latex rubber or silicon, which are attached together and pointing towards the second end of the urinary catheter. When the urine reaches the first cone shaped valve in the capsular segment, due to the force of the liquid flow and weight of the liquid, the tip of the first cone shaped valve opens allowing the urine to pass through the opening. Similarly the second and third cone shaped valves are opened to allow the urine to pass through the valves to the collection bag located outside the urinary catheter. Thus the collected urine in the collection bag cannot re-enter the human body due to the presence of one way cone shaped valves in the capsular segment of the urinary catheter device.

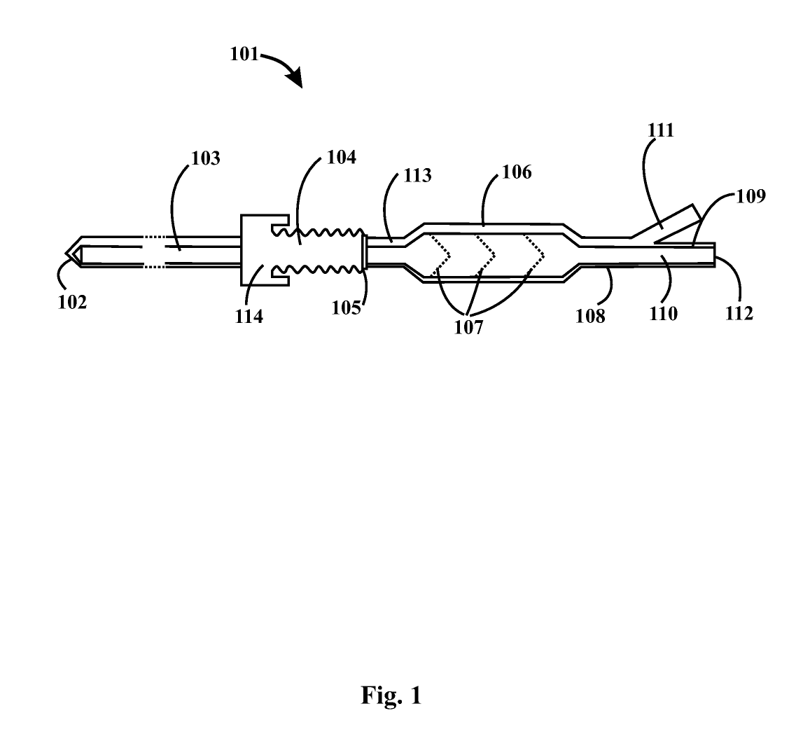

FIG. 1 illustrates a top side perspective view of the urinary catheter device 101 used in draining out urine from the bladder according to an embodiment of the present disclosure. The urinary catheter 101 includes a body having an interior surface 110 and an exterior surface 109. The exterior surface 109 of the urinary catheter 101 is made of a flexible material such as rubber whereas the interior surface 110 of the urinary catheter is made of materials such as silicon or latex rubber.

The body 108 of the urinary catheter 101 extends from a first end 102 of the urinary catheter 101 to the second end 112 . The first end 102 is connected to the urinary bladder of the patient and the second end 112 is connected to the collection bag to collect the drained urine. The second end 112 of the urinary catheter is connected to the collection bag using a plastic tubular pipe.

The first end 102 of the catheter body 108 includes a rounded end section to facilitate insertion of the urinary catheter 101 into the urinary bladder of the patients. The interior surface 110 is formed as a narrow way within the catheter body 108 which allows flow of fluid from the body cavity, for instance, the urinary bladder to enter the hollow passageway which connects the interior surface 110 of the urinary catheter 101 from the first end 111 to the second end 112.

The urinary catheter 101 further includes a vaginal condom segment 104 to connect the urinary catheter 101 to at least one of the glands of penis in men and around the vagina in women. This process is accomplished with the help of a sticky part on the edge of the convexity of the crescent segment 114 of the vaginal-condom segment 104. The urine flows from the patient to the collection bag through a hollow passageway and enters the capsular segment 106 in the urinary catheter 101. The capsular segment 106 is a capsule shaped part within the interior surface 110 of the urinary catheter 101 and located adjacent to the second end 112.

The capsular segment 106 comprises of at least three one way valves 107. As the three one way valves 105 are made of two pieces of separable and flexible plastic, the force and weight of the urine liquid entering into the capsular segment 106 results in opening of the first of the three one way valves 105 thereby allowing the urine to flow through the valves. Similarly, the force and weight of urine liquid causes the second and third valves of the three one way valves 105 to open allowing the urine to flow towards the collection bag. Once the urine reaches the collection bag, the re-entry of the drained urine liquid to the patient's body is avoided as the vales are constructed in such a way that the passing of urine liquid is unidirectional through the capsular segment 106. The urinary catheter 101 further includes a stopper 105 which is a small tuber structure located near the capsular segment 106 of the urinary catheter 101. The stopper 105 connects the vaginal condom segment 104 on to the urinary catheter 101 and prevents the detachment of the vaginal condom segment from the urinary catheter 101.

The embodiment according to FIG. 1 also illustrates an inflation lumen for balloon at the first end 102 of the urinary catheter 101 used in conventional urinary catheters 101.

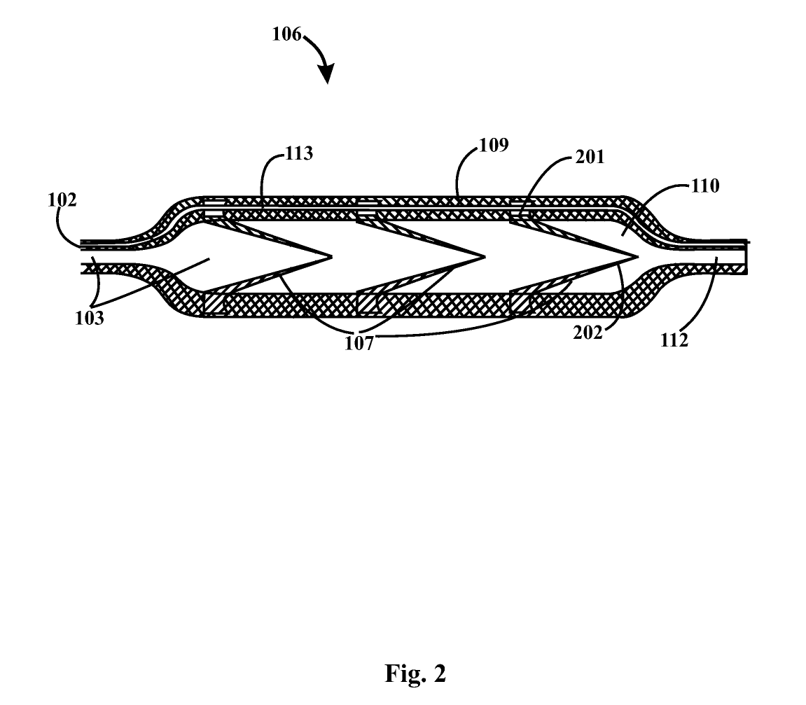

FIG. 2 illustrates a sectional view of the capsular segment 106 and a three one way cone shaped valves 107 arranged in close contact position according to an embodiment of the present disclosure. The capsular segment 106 of the urinary catheter comprises of three one way cone shaped valves 107 which are of single flexible or elastic material made of latex rubber or silicon. The flow of urine through the hollow passage way 103, exerts a force on the first of the three one way cone shaped valves 107 in the capsular segment 106. The three one way cone shaped valves 107 located in the capsular segment 106 of the urinary catheter 101 include a valve floor 201 which is of infundibular form and narrowing towards the second end 112 of the urinary catheter. The valve tip 202 is made of two separable plates made of latex rubber or silicon, sticking together and pointing towards the second end 112 of the urinary catheter. When the urine reaches the first cone shaped valve 107 in the capsular segment 106, due to the force of the liquid flow and weight of the liquid, the valve tip 202 of the first cone shaped valve 107 opens allowing the urine to pass through the opening. Similarly the second and third cone shaped valve 107 opens and allows the urine to pass through the valve to the collection bag located outside the urinary catheter 101. The urine collected in the collection bag cannot re-enter the human body due to the presence of one way cone shaped valves 107 in the capsular segment 106 of the urinary catheter device.

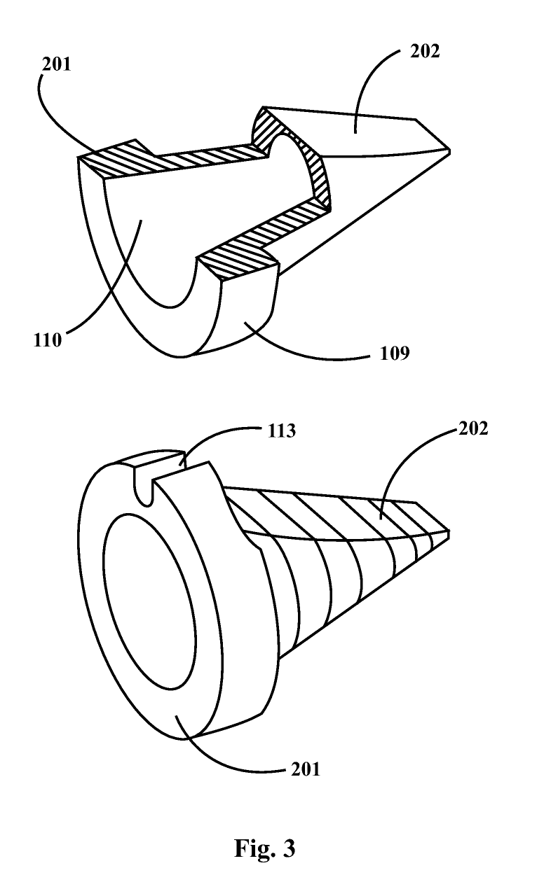

FIG. 3 illustrates a sectional view of one way valves inside the capsular segment of the urinary catheter according to an embodiment of the present disclosure. The valve includes a valve floor 201 and a valve tip 202. The valve floor 201 is in infundibular form and is extended towards the first end of the urinary catheter. The valve tip 202 includes at least two plates that are attached together and is pointed towards the second end of the urinary catheter. When the urine reaches valve tip 202 of the cone shaped valves located in the capsular segment through the valve floor 201, due to the force and the direction of the liquid flow, the at least two plates of the valve tip 202 opens to allow the flow of urine thorough the valve.

The same process of operation is carried out with the second and third valves present in the capsular segment. The valves also show balloon inflation passage way 113 through which the lumen of the balloon was controlled in conventional urinary catheters.

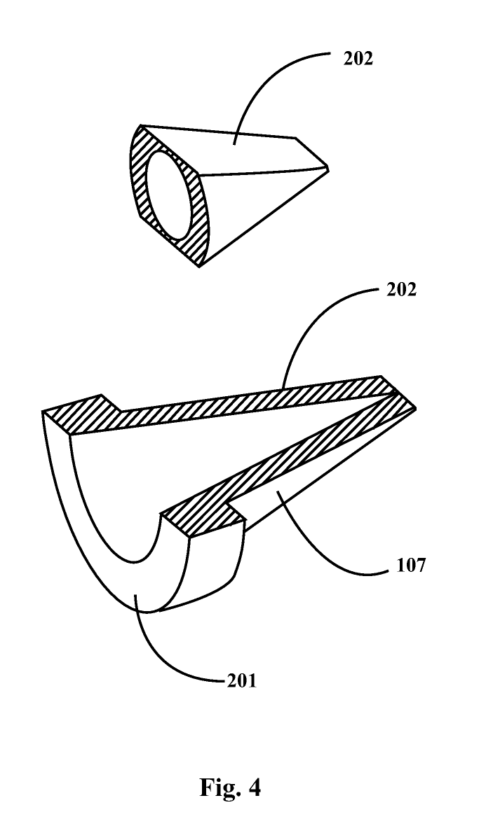

FIG. 4 illustrates a sectional view of the tip of the valve 202 and the valve floor 201 present in capsular segment 106 of the urinary catheter device according to an embodiment of the present disclosure. The three one way cone shaped valves 107 are located within the capsular segment 106 and including the valve floor 201 which is in infundibular form and pointing towards the first end 102 of the catheter 101. The valve tip 202 includes two plates that are stick together and narrowing towards the second end 112 of the urinary catheter 101. When the urine enters the capsular segment 106, the force and direction of the urine flow separates the two plates, which is valve tip 202 allowing the urine to pass through the three one way cone shaped valves 107. The three one way cone shaped valves 107 are constructed of the same material which is used in the construction of the urinary catheter 101 which is latex rubber or silicon. The three one way cone-shaped valves 107 are connected to the interior surface 110 of the urinary catheter and three one way valves 107 are arranged with the same distance between each valve within the capsular segment 106. The fluid flow starts from the infundibular part of the valve floor 201 and enters to the valve tip 202 and opens the valves. The thickness of the three one way valves 107 is decreased from the valve floor 201 to the valve tip 202, thereby making the valves thicker at the valve floor 201 and thinner at the valve tip 202. This prevents the back flow of the urine to the human body.

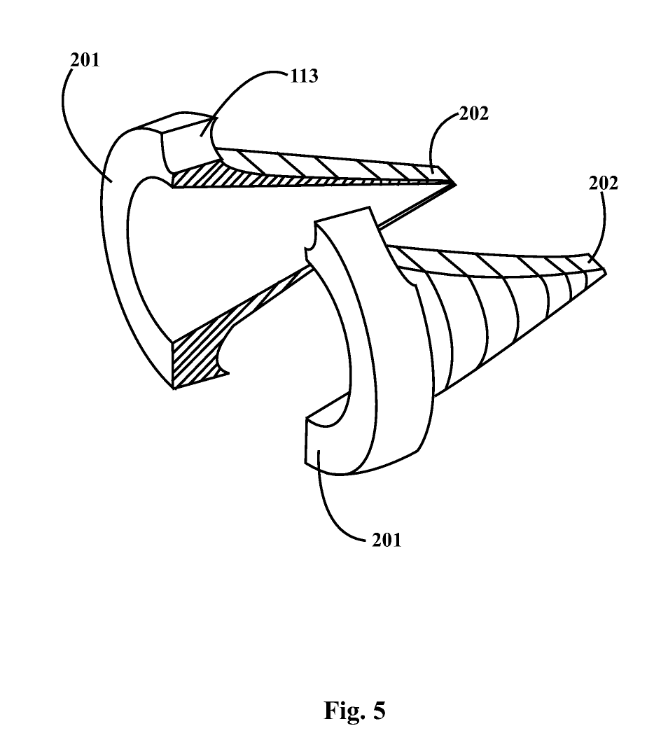

FIG. 5 illustrates the exploded sectional view of the one of the three one way valves 107 in the capsular segment 106 of the urinary catheter 101 device according to an embodiment of the present disclosure. The three one way valves 107 are placed inside the capsular segment 106. The three one way valves include a valve floor 201 which is in infundibular form and pointing towards the first end 102 of the catheter 101. Further the thickness of the three one way valves 107 is decreased from the valve floor 201 to the valve tip 202 making thicker at the valve floor 201 and thinner at the valve tip 202. Also shown is a balloon inflation passage 113 where a balloon was used with the catheter to connect the catheter to a patient.

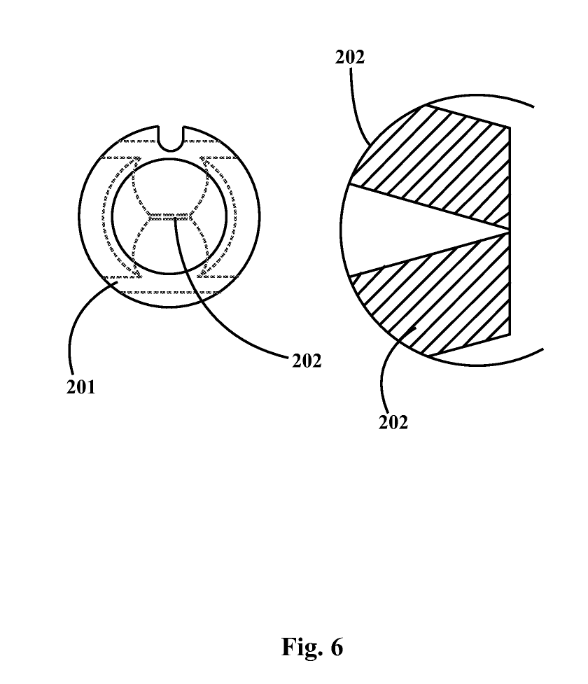

FIG. 6 illustrates a top view and a sectional view of one of the three one way cone shaped valve in the capsular segment of the urinary catheter according to an embodiment of the present disclosure. The valve of the catheter comprises of a valve floor 201 and a valve tip 202 that is in infundibular form and pointing towards the first end 102 of the catheter. Further the thickness of the three one way valves is decreased from the valve floor 201 to the valve tip 202 making thicker at the valve floor 201 and thinner at the valve tip 202.

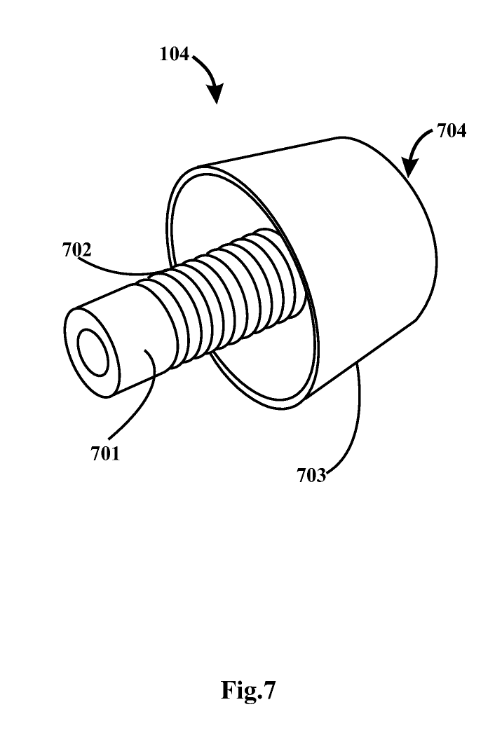

FIG. 7 illustrates a perspective view of the vaginal condom segment 104 attached to the urinary catheter according to an embodiment of the present disclosure. The vaginal condom segment 104 includes a unique design and is adapted to connect the urinary catheter to the human body. The vaginal condom segment 104 is comprised of a crescent segment 703 and a flexible arm 701. The flexible arm 701 includes a plurality of folds 702 to connect the catheter into the human body without any intervention. The plurality of folds are expandable and contractible folds 702 which provides for varying the length of the vaginal condom segment 104 depending upon the sexuality of the patients.

The crescent segment 703 is located at the initial stage of the vaginal-condom segment 104 facing the second end of the urinary catheter. The vaginal-condom segment 104 is used to connect the urinary catheter to glands of penis in men and around vagina in women. This process is accomplished with the help of a sticky part on the edge of the convexity 704 of the crescent segment 703 of the vaginal-condom segment 104.

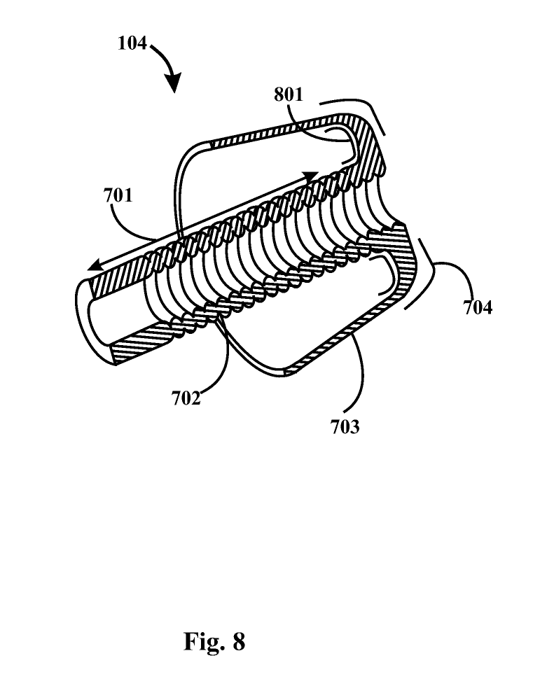

FIG. 8 illustrates a side cross sectional view of the vaginal condom segment according to an embodiment of the present disclosure. The vaginal condom segment 104 includes a flexible arm 701 and a crescent segment 703. The vaginal condom segment 104 is made of unique design including a plurality of folds 702 present on the flexible arm 701 which helps in connecting the catheter into the human body without any intervention. The length of the flexible arm 701 is variable manually depending upon the sexuality of patients as the flexible arm 701 is made up of one or more expandable and contractable folds. A crescent segment 703 is located at the initial stage of the vaginal-condom segment 104 such that the concave edge 801 is facing the first end 102 and convexity 704 is facing the second end of the urinary catheter. The vaginal-condom segment 104 is adapted to connect the urinary catheter to the glands of penis in men and around the vagina in women.

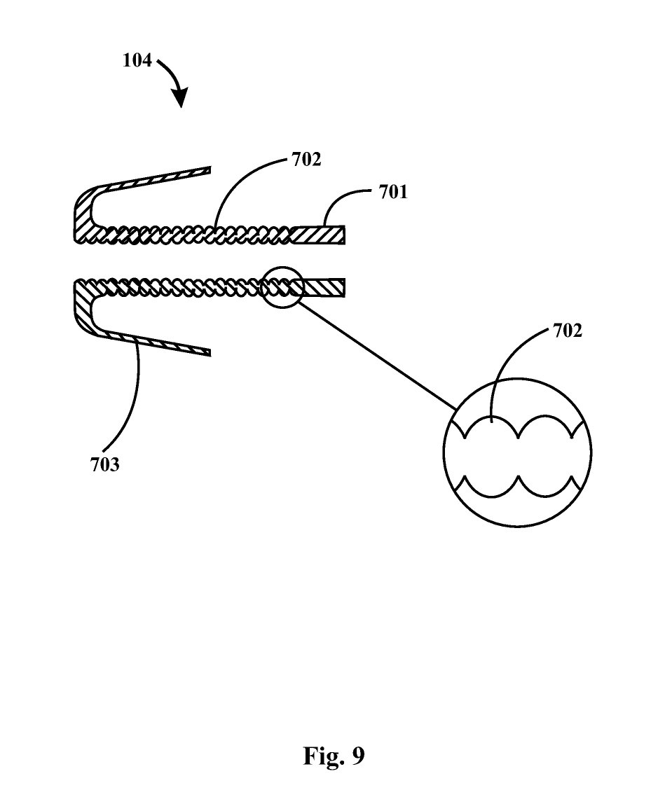

FIG. 9 illustrates a side cross sectional view of the vaginal condom segment 104 with folds 702, attached to the urinary catheter device according to an embodiment of the present disclosure. The vaginal condom segment 104 is designed to connect the urinary catheter to the glands of penis in men and to connect around the vaginal opening in women. The vaginal-condom segment 104 includes a flexible arm 701. The flexible arm 701 helps to variably increase or decrease the length of the vaginal condom segment 104 in proportionate with the sexuality of the patients. The flexible arm 701 includes a plurality of folds which provides for varying the length of the vaginal condom segment 104. The vaginal condom segment 104 further includes a crescent segment 703 located at a starting section of the vaginal condom segment 104 along a second end of the urinary catheter.

The crescent segment 703 and the flexible arm 701 stick together to form the vaginal condom segment 104 component of the urinary catheter. The crescent segment 703 includes a sticky part that located on the convex edge of the crescent segment 703 which causes the vaginal-condom segment 104 to stick on closely with the vaginal opening in women and to stick on to the glands of penis in men. The crescent segment 703 of the vaginal condom segment 104 to stick on to the glands of penis in men, the convexity 704 of the crescent segment is facing towards the second end of the crescent segment 703. The crescent segment 703 when placed on the vaginal opening of the women, the concavity 801 of the crescent segment 703 is facing towards the second end and edge of the convexity 704 of the crescent segment 703 is placed on the vaginal opening in women.

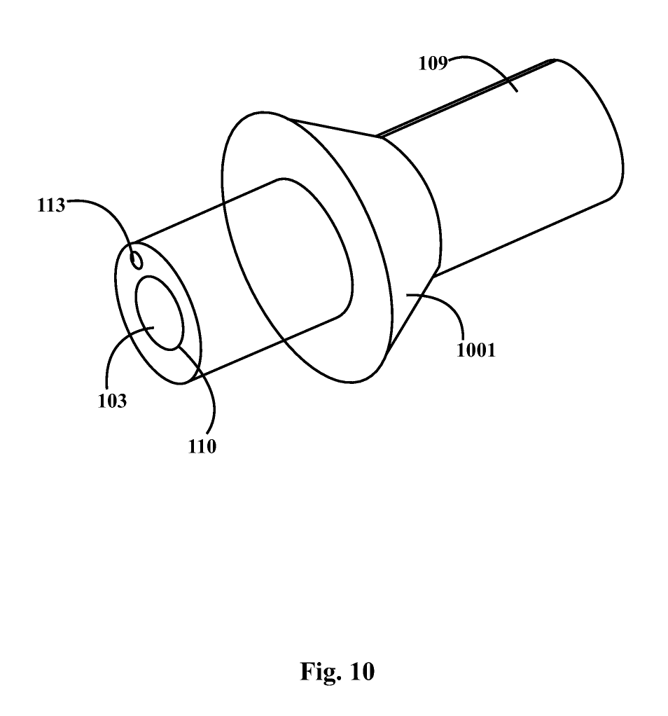

FIG. 10 is a schematic view of the urinary catheter illustrating a stopper which prevents a detachment of the vaginal condom segment from the urinary catheter according to an embodiment of the present disclosure.

The stopper 1001 is a tuber of the exterior surface 109 of the urinary catheter and adjacent to the capsular segment of the urinary catheter. The flexible arm part of the vaginal condom segment is adapted to be in contact with the stopper 1001 which prevents the separation of the vaginal condom segment from the urinary catheter. The stopper 1001 attached to the urinary catheter eliminates the need for the balloon to keep the urinary catheter 101 connected to the patient's body.

According to one embodiment of the present disclosure, the cone shaped valves includes a unique design which causes a unilateral routing of the urine from the urinary bladder towards the collection bag. This unilateral routing prevents a urinary reflux flow which in turn prevents the entry of the harmful microorganisms into the patient's bladder. The prevention of the urinary reflux also avoids the re-entry of the drained materials into the patients' bladder, resulting in a reduction of Catheter Associated Urinary Tract Infections in the patients.

Further, due to the presence of one way cone shaped valves, the patient's intervention is not required in managing the drained fluids from the bladder as the one way cone shaped valves does not allow the re-entry of the drained fluids. The vaginal condom segment is designed such that the catheter can be used to connect the glands of penis in men and the vaginal opening in women making the catheter device usage common for the patients of both the genders. Another embodiment of the disclosure is the stopper that is integrated to the exterior surface of the urinary catheter. The stopper eliminates the need for the balloon which is used in conventional catheters.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A capsular urinary catheter to manage urinary incontinence or retention in patients, the urinary catheter comprising:
a first end adapted to be inserted into the patient's body;
a second end adapted to be releasably attached to a fluid collection reservoir;
a body defining both an exterior surface and an interior surface;
a vaginal condom segment attached to the exterior surface;
a capsular segment defining a capsule in the interior surface;
three one way valves arranged inside the capsular segment;
wherein the three one way valves includes at least two plates which gets separated due to a force exerted by a fluid movement, thereby causing the passage of a fluid from the first end towards the second end and prevents a fluid reflux from the second end towards the first end of the catheter.

2. The capsular urinary catheter according to claim 1, wherein the interior surface defines a central lumen through which a urine flows from a urinary bladder to the fluid collection reservoir.

3. The capsular urinary catheter according to claim 1, wherein the body segment and the three one way valves are made of a flexible plastic material such as latex or silicone.

4. The capsular urinary catheter according to claim 1, wherein the three one way valves are cone-shaped such that a first section of the cone-shaped valve binds within the interior surface of the catheter.

5. The capsular urinary catheter according to claim 1, wherein the three one way valves comprises:
a infundibular valve floor extending towards the first end of the catheter;
and a valve tip extending towards the second end of the catheter.

6. The capsular urinary catheter according to claim 1, wherein the valve tip includes two seperably attached plates tapering towards the second end of the urinary catheter.

7. The capsular urinary catheter according to claim 1, wherein the two plates of the valve tip moves away from each other due to a force exerted by the urine flow from the urinary bladder thereby opening the three one way valves to direct the urine flow to the collection reservoir.

8. The capsular urinary catheter according to claim 1, wherein the two plates stick together due to the elasticity of the material resulting in the closure of a fluid flow route thereby preventing the urine reflux.

9. The capsular urinary catheter according to claim 1, wherein the three one way valves are arranged equidistantly within the capsular segment.

10. The capsular urinary catheter according to claim 1, the vaginal condom segment is adapted to connect the urinary catheter with a glands of penis in men and around a vagina in women.

11. The capsular urinary catheter according to claim 1, comprising:
a cresent segment arranged adjacent to a beginning part of the vaginal condom segment; and a flexible arm.

12. The capsular urinary catheter according to claim 11, wherein the flexible arm includes a plurality of folds which provides for varying the length of the flexible arm in proportionate with a sexuality of the patients.

13. The capsular urinary catheter according to claim 11, wherein the crescent segment is removable towards at least one of the first end and the second end of the catheter.

14. The capsular urinary catheter according to claim 11, wherein the crescent segment includes a convexity part and a concavity part to place the crescent segment in proportionate with the sexuality of the patients.

15. The capsular urinary catheter to claim 15, wherein the crescent segment is placed such that a concavity part in women is towards the second end of the catheter and the convexity part on the vaginal opening in women.

16. The capsular urinary catheter to claim 11, wherein the crescent segment is placed such that the convexity part in men is towards the second end of the catheter and the concavity part on the vaginal opening in women.

17. The capsular urinary catheter to claim 11, wherein the crescent segment includes a sticky part located on an edge of convexity part to stick on to the vaginal opening in women and the glands of penis in men.

18. The capsular urinary catheter to claim 11, wherein the crescent segment is of a sticky-plaster type to stick to the crescent segment from one side and to the vaginal opening in women from the other side.

19. The capsular urinary catheter to claim 11, wherein the crescent segment is of a sticky-plaster type to stick to the crescent segment from one side and to the glands of penis in men from the other side.

20. The capsular urinary catheter according to claim 1, further comprising a stopper placed on the exterior surface to prevent a separation of the vaginal condom segment from the catheter.
